(19) United States
(12) Patent Application Publication
(10) Pub. No.: US 2004/0241173 A1
(43) Pub. Date: Dec. 2, 2004

(54) Fullerene (C60) vancomycin conjugates as improved antibiotics

(75) Inventors: Lon J. Wilson, Houston, TX (US); Andrey L. Mirakyan, Houston, TX (US); Matthew P. Cubbage, New York, NY (US)

Correspondence Address: CONLEY ROSE, P.C., P. O. BOX 3267, HOUSTON, TX, 77253-3267, US

(73) Assignee: William Marsh Rice University, Houston, TX (US)

(21) Appl. No.: 10/776,844

(22) Filed: Feb. 11, 2004

Related U.S. Application Data
(63) Continuation-in-part of application No. 10/367,646, filed on Feb. 14, 2003.
(63) Continuation-in-part of application No. 10/623,110, filed on Jul. 18, 2003.
(63) Continuation-in-part of application No. 10/623,190, filed on Jul. 18, 2003.
(60) Provisional application No. 60/446,406, filed on Feb. 11, 2003.
(60) Provisional application No. 60/356,856, filed on Feb. 14, 2002.

(51) Int. Cl. A61K 39/395; C07K 16/46

(52) U.S. Cl. 424/178.1; 530/391.1; 424/125; 514/8; 514/190; 514/200; 514/33; 514/152; 514/252.13; 514/253.08; 514/312; 530/322; 536/18.1; 540/222; 540/304; 544/363; 546/156

(57) ABSTRACT

A fullerene-antibiotic conjugate including at least one antibiotic molecule per fullerene moiety. The fullerene may comprise C₆₀ and the antibiotic may comprise vancomycin or may be selected from the group consisting of penicillins, cephalosporins, quinolones, fluoroquinolones, macrolides, lincosamines, carbepenems, conobactams, aminoglycosides, glycopeptides, tetracyclines, sulfonamides, rifampin, oxazolidonones, and streptogramins. The conjugate preferably includes at least two and more preferably at least three antibiotic molecules per C₆₀ center. A method for making a fullerene(C₆₀)-antibiotic conjugate, comprises: synthesizing a linker precursor (I); reacting the linker precursor (I) with C₆₀ via a Bingel-reaction, to produce a fullerene-linker conjugate (II); hydrolyzing the fullerene-linker conjugate (II), resulting in a desired derivative of C₆₀ (III); and reacting the derivative (III) with a desired antibiotic to produce a fullerene-antibiotic conjugate (IV).

### CROSS-REFERENCE TO RELATED APPLICATIONS

This application claims priority from provisional application 60/446,406, filed Feb. 11, 2003, and is also a continuation in part of U.S. application Ser. No. 10/367,646, filed Feb. 14, 2003, which claims priority from provisional application 60/356,856, filed Feb. 14, 2002, and is also a continuation in part of U.S. application Ser. Nos. 10/623,110 and 10/623,190, both filed on Jul. 18, 2003, all of which are hereby incorporated by reference in their entireties.

### STATEMENT REGARDING FEDERALLY SPONSORED RESEARCH OR DEVELOPMENT

Not Applicable.

### FIELD OF THE INVENTION

The present invention relates generally to fullerenes conjugated with bioactive agents and more particularly to fullerenes conjugated with antibiotics and targeting agents.

### BACKGROUND OF THE INVENTION

Antibiotics are widely used to treat bacterial infections. For example, the antibiotic vancomycin is a relatively small glycoprotein (MW˜1,450) derived from Nocardia Orientalis. Its chemical structure is illustrated in FIG. 1. Vancomycin is active against most G(+) bacteria including Streptococci, Corynebacteria, Clostridia, Listeria, and Bacillus species. Vancomycin is presently the antibiotic of last resort in many instances. Unfortunately, now, forty years after the drug was first introduced into the clinic, bacteria are becoming resistant to vancomycin.

Similar issues have arisen in the use of other antibiotics. As bacterial resistance grows, more powerful antibiotics have been developed, but these may have stronger side effects than less powerful antibiotics. Hence, there is a need for antibiotics to which bacteria are less resistant. There is also a need for ways to reduce patient exposure to the antibiotic, while still providing sufficient antibiotic to ensure effective treatment.

### SUMMARY OF THE INVENTION

The present invention provides antibiotics to which bacteria are less resistant. In addition, by enabling the antibiotic to be targeted to a desired site, the present invention allows the use of smaller dosages, thereby reducing patient exposure to the antibiotic while still providing sufficient antibiotic to ensure effective treatment.

In certain embodiments, the present invention includes a chemical modification of vancomycin or another antibiotic that may lead to increased antibacterial activity against resistant strains and may provide the ability to reduce dosages by enabling the administered compound to be targeted to a desired location or agent. This chemical modification involves the synthesis of new fullerene(C₆₀)-antibiotic conjugates, containing one or more antibiotic molecules per C₆₀ center. It is expected that the antibacterial activity of vancomycin and other antibiotics such as penicillin, cephalosporins, fluoroquinolones etc. can be improved as fullerene(C₆₀)-antibiotic conjugates.

It is believed that the present fullerene-antibiotic conjugates can be particularly effective if they are conjugated further with one or more targeting agents that will bind the conjugate at or near the desired treatment site. In some preferred embodiments, the fullerene is C₆₀ and the antibiotic is vanomycin. In other embodiments, the fullerene and/or the antibiotic may be varied.

In certain preferred embodiments, fullerene-vancomycin conjugates are used to treat osteomyelitis, by further derivatization with diphosphonate groups to target the antibiotic to bone. In other embodiments, fullerene-antibiotic conjugates are target to bacterial cell walls or bacterial spores and are administered to a patient via an aerosol spray.

Thus, the present invention comprises a combination of features and advantages that enable it to overcome various problems associated with un-conjugated antibiotics. The various characteristics described above, as well as other features, will be readily apparent to those skilled in the art upon reading the following detailed description of the preferred embodiments of the invention, and by referring to the accompanying drawings.

### BRIEF DESCRIPTION OF THE DRAWINGS

For a more detailed description of the preferred embodiment of the present invention, reference will now be made to the accompanying drawings, wherein:

FIG. 1 is a chemical diagram of a molecule of vancomycin hydrochloride, C₆₆H₇₅Cl₂N₉O₂₄.HCl; and

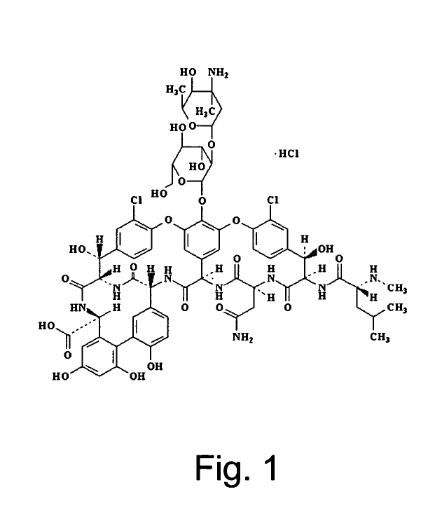

FIGS. 2(A)-(D) are schematic chemical diagrams of one reaction series that can be used to generate a fullerene-antibiotic conjugate in accordance with the present invention.

### DETAILED DESCRIPTION OF THE PREFERRED EMBODIMENT

The present invention comprises fullerene-antibiotic conjugates that can be used as new same-class antibiotics for the treatment of bacteria strains that are resistant to the unconjugated antibiotic. More particularly, we suggest herein a novel method for producing compounds that include several antibiotic units per molecule. The number of antibiotic units can be controlled by design of the initial fullerene derivative. This is expected to enhance efficacy of the antibiotic. In some embodiments, attachment of a C₆₀ moiety to an antibiotic may allow the resulting conjugate to enter cells, thereby expanding the antibiotic activity to intracellular bacteria. In other embodiments, a targeting agent comprising one or more antibodies is attached to the fullerene-antibiotic conjugate.

One preferred method for forming a fullerene-antibiotic conjugate can be generalized as including two steps. Namely, 1) the synthesis of a fullerene derivative containing one or more linkers and 2) condensation of the fullerene derivative obtained in the step 1 with an antibiotic or other bioactive agent. In certain embodiments, these steps can be accomplished by forming a malonate or other linking molecule with protected ends, attaching the linking molecule to a fullerene, removing the protective groups from the end(s) of the linking molecule, and affixing the desired bioactive agent to the end(s) of the linking molecule. An illustrative reaction scheme is set out in the Example and discussed in detail below.

While the Example illustrates an embodiment comprising vancomycin, other antibiotics, including particularly those containing a carboxyl group (i.e. cipro) can be attached to C₆₀ in a similar manner. Thus, other embodiments involving other antibiotics include the following:

ample spectrum penicillins, including amoxicillin, ampicillin, bacampicillin, carbenicillin indanyl, mezlocillin, piperacillin, ticarcillin;

penicillins and beta lactamase inhibitors, including amoxicillin-clavulanic acid, ampicillin-sulbactam, benzylpenicillin, cloxacillin, dicloxacillin, methicillin, oxacillin, penicillin G (benzathine, potassium, procaine), penicillin v, piperacillin+tazobactam, ticarcillin+clavulanic acid, nafcillin;

cephalosporins I-IV generations;

macrolides and lincosamines, including azithromycin, clarithromycin, clindamycin, dirithromycin, erythromycin, lincomycin, troleandomycin;

quinolones and fluoroquinolones, including cinoxacin, ciprofloxacin, enoxacin, gatifloxacin, grepafloxacin, levofloxacin, lomefloxacin, moxifloxacin, nalidixic acid, norfloxacin, ofloxacin, sparfloxacin, trovafloxacin, oxolinic acid, gemifloxacin, perfloxacin

carbepenems, including imipenem-cilastatin, meropenem;

conobactams, including aztreonam;

aminoglycosides, including amikacin, gentamicin, kanamycin, neomycin, netilmicin, streptomycin, tobramycin, paromomycin;

glycopeptides, including teicoplanin, vancomycin;

tetracyclines, including demeclocycline, doxycycline, methacycline, minocycline, oxytetracycline, tetracycline, chlortetracycline;

sulfonamides, including mafenide, silver sulfadiazine, sulfacetamide, sulfadiazine, sulfamethoxazole, sulfasalazine, sulfisoxazole, trimethoprim-sulfamethoxazole, sulfamethizole;

rifampin, including rifabutin, rifampin, rifapentine;

oxazolidonones, including linezolid;

streptogramins, including quinopristin+dalfopristin;

and others, including bacitracin, chloramphenicol, colistemetate, fosfomycin, isoniazid, methenamine, metronidazol, mupirocin, nitrofurantoin, nitrofurazone, novobiocin, polymyxin b, spectinomycin, trimethoprim, colistin, cycloserine, capreomycin, ethionamide, pyrazinamide, para-aminosalicyclic acid, erythromycin ethylsuccinate+sulfisoxazole.

Similarly, while a preferred embodiment comprising C₆₀ is described herein, fullerenes suitable for use in the present invention include C₆₀, C₇₀, C₈₀, C₁₂₀, C₂₄₀, endofullerenes, nanotubes, higher carbon number fullerenes, and derivatives thereof.

Likewise, other synthetic approaches different from the one described here could also be used to attach vancomycin or other antibiotics to fullerenes. Suitable techniques will be recognizable to those having ordinary skill in the art of organic chemistry. A bone-targeted antibiotic might also be prepared using a similar reaction scheme, by condensing vancomycin or other suitable antibiotic with an aminodiphosphonate derivative, such as 3-amino propylenediphosphonic acid.

If desired, a targeting agent can also be attached to the fullerene molecule, either before or after the antibiotic. The targeting agent may be a protein or an antibody, such as a glycogen IIa/IIB receptor antibody, Von Willebrand's factor antibody, an antitumor antibody, hepatic cellular antibody, a white blood cell antibody, or antifibrin, as described in U.S. Pat. No. 6,352,683, which is incorporated herein by reference.

In certain embodiments, the targeting agent can be a moiety comprising an antigen-binding site. An “antigen,” as used herein, is a chemical compound or a portion of a chemical compound which can be recognized by a specific chemical reaction, a specific physical reaction, or both with another molecule. The antigen-recognition site of an antibody is an exemplary, but non-limiting, antigen-binding site. Examples of moieties comprising antigen-binding sites include, but are not limited to, monoclonal antibodies, polyclonal antibodies, Fab fragments of monoclonal antibodies, Fab fragments of polyclonal antibodies, Fab₂ fragments of monoclonal antibodies, and Fab₂ fragments of polyclonal antibodies, among others. Single chain or multiple chain antigen-recognition sites can be used. Multiple chain antigen-recognition sites can be fused, joined by a linker, or unfused and unlinked.

The targeting agent can be selected from any known class of antibodies. Known classes of antibodies include, but are not necessarily limited to, IgG, IgM, IgA, IgD, and IgE. The various classes also can have subclasses. For example, known subclasses of the IgG class include, but are not necessarily limited to, IgG1, IgG2, IgG3, and IgG4. Other classes have subclasses that are routinely known by one of ordinary skill in the art.

Similarly, the targeting agent can be derived from any species. “Derived from,” in this context, can mean either prepared and extracted in vivo from an individual member of a species, or prepared by known biotechnological techniques from a nucleic acid molecule encoding, in whole or part, an antibody peptide comprising invariant regions which are substantially identical to antibodies prepared in vivo from an individual member of the species or an antibody peptide recognized by antisera specifically raised against antibodies from the species. Exemplary species include, but are not limited to, human, chimpanzee, baboon, other primate, mouse, rat, goat, sheep, and rabbit, among others known in the art. In one embodiment, the targeting agent is chimeric, i.e., comprises a plurality of portions, wherein each portion is derived from a different species. A chimeric antibody, wherein one of the portions is derived from human, can be considered a humanized antibody.

Targeting agents are available that recognize antigens associated with a wide variety of cell types, tissues, and organs, and a wide variety of medical conditions, in a wide variety of mammalian species. Exemplary medical conditions include, but are not limited to, cancers, such as lung cancer, oral cancer, skin cancer, stomach cancer, colon cancer, nervous system cancer, leukemia, breast cancer, cervical cancer, prostate cancer, and testicular cancer; arthritis; infections, such as bacterial, viral, fungal, or other microbial infections; and disorders of the skin, the eye, the vascular system, or other cell types, tissues, or organs; among others.

Exemplary targeting agents include, but are not limited to, those derived from antibodies against anthrax or other bacteria, antibodies against the spores of anthrax or other bacteria, antibodies against vascular endothelial growth factor receptor (VEGF-r) (available from Imclone, New York, N.Y.), antibodies against epidermal growth factor receptor (EGF-r) (available from Abgenix, Fremont, Calif.), antibodies against polypeptides associated with lung cancers (available from Corixa Corporation, Seattle, Wash.), and antibodies against human tumor necrosis factor alpha (hTNF-α) (available from BASF A.G., Ludwigshafen, Germany), among others known in the art. Exemplary targeting agents suitable for use against sporulating microbes are disclosed in U.S. Pat. No. 5,510,104, which is incorporated herein by reference.

Suitable targeting agents can be prepared by various techniques that are known in the art. These techniques include, but are not limited to, the immunological technique described by Kohler and Milstein in Nature 256, 495-497 (1975) and Campbell in “Monoclonal Antibody Technology, The Production and Characterization of Rodent and Human Hybridomas” in Burdon et al., Eds., Laboratory Techniques in Biochemistry and Molecular Biology, Volume 13, Elsevier Science Publishers, Amsterdam (1985); as well as by the recombinant DNA techniques described by Huse et al in Science 246, 1275-1281 (1989); among other techniques known to one of ordinary skill in the art.

In addition to the listed antibodies, the targeting agent can be constructed to recognize a target antigen associated with a solid tumor. For example, the targeting agent can be constructed to recognize HER2/neu, MUC-1, HMFG1, or EGFr, associated with breast tumors; MMP-9, HER2/neu, or NCAM, associated with lung tumors; HER2 or 171A, associated with colon tumors; gp240, gangliosides, or integrins, associated with melanomas; HER2 or CA-125, associated with ovarian tumors; or EGFr or tenascin, associated with brain tumors. This list of target antigens and tumor types is exemplary and not limiting.

Compounds made in accordance with the present invention can be used to treat disease. For example, an infection caused by a microorganism can be treated by contacting the microorganism with a fullerene-antibiotic conjugate. If the infection is localized to a particular tissue type or site, a targeting agent can be used to ensure that the fullerene-antibiotic conjugate be likewise localized. For example, the targeting agent may be a bone-targeting moiety or bacteria-targeting moiety as described above. Use of a targeting agent may reduce unnecessary exposure of the rest of the patient to the antibiotic.

If desired, the fullerene-antibiotic conjugate may be provided in a pharmaceutically acceptable carrier. The desired antibiotic effect may be achieved by administering to the patient a pharmaceutically acceptable composition containing fullerene-antibiotic conjugate in a dose effective to inhibit the growth of a bacterial species in the patient. As described herein, the fullerene-antibiotic conjugate may comprise C₆₀ conjugated with an antibiotic is selected from the group consisting of penicillins, cephalosporins, quinolones, fluoroquinolones, macrolides, lincosamines, carbepenems, conobactams, aminoglycosides, glycopeptides, tetracyclines, sulfonamides, rifampin, oxazolidonones, and streptogramins, with or without a targeting agent.

Delivery of the present pharmaceutical compositions may be made by any suitable technique, including but not limited to: non-systemic delivery routes, including colonic delivery routes, ingestive delivery routes, topical applications of cream, gel, or ointment, and systemic delivery routes, including ingestion, injection, intravenous drip, implant, transdermal delivery routes, and transmucosal delivery routes.

In certain preferred embodiments, the present compositions are administered via an inhaled aerosol spray. For example, the present compounds can be used in the treatment of patients who have been exposed to anthrax and/or other weaponized bacteria. In these embodiments, the targeting agent is preferably capable of binding to bacterial spores. The spore stage of the microbial life cycle is characterized by metabolic dormancy and resistance to environmental factors that would destroy the microbe in its vegetative stage. The earliest phase of spore germination is characterized by swelling and a shift from dormancy to active metabolism. Sprouting, and reproduction follow. Hence, it is desirable to attack microbes while they are in the spore stage. The targeted fullerene-antibiotic composition may be administered to the patient in an aerosol mist or spray that is inhaled and contacts the spores.

In still other embodiments, the targeted fullerene-conjugate is configured to bind to the cell walls of targeted microbes.

The present procedure is described and shown for the case of a monoadduct, but up to 8 malonate groups can be placed on C₆₀ using the same sequence of reactions. In some instances, the attachment of multiple antibiotics is preferred. For example, conjugates containing less than three molecules of vancomycin per C₆₀ may have low solubility in water. Attachment of other hydrophilic groups (malonate, serinol etc.) or further derivatization of C₆₀ with more vancomycin are expected to provide sufficient water solubility of the resulting conjugates. Other reaction schemes by which conjugation may be carried out are described in U.S. Pat. No. 6,660,248 B2, entitled “Fullerene (C₆₀)-Based X-Ray Contrast Agent for Diagnostic Imaging,” which is incorporated herein by reference.

### EXAMPLE

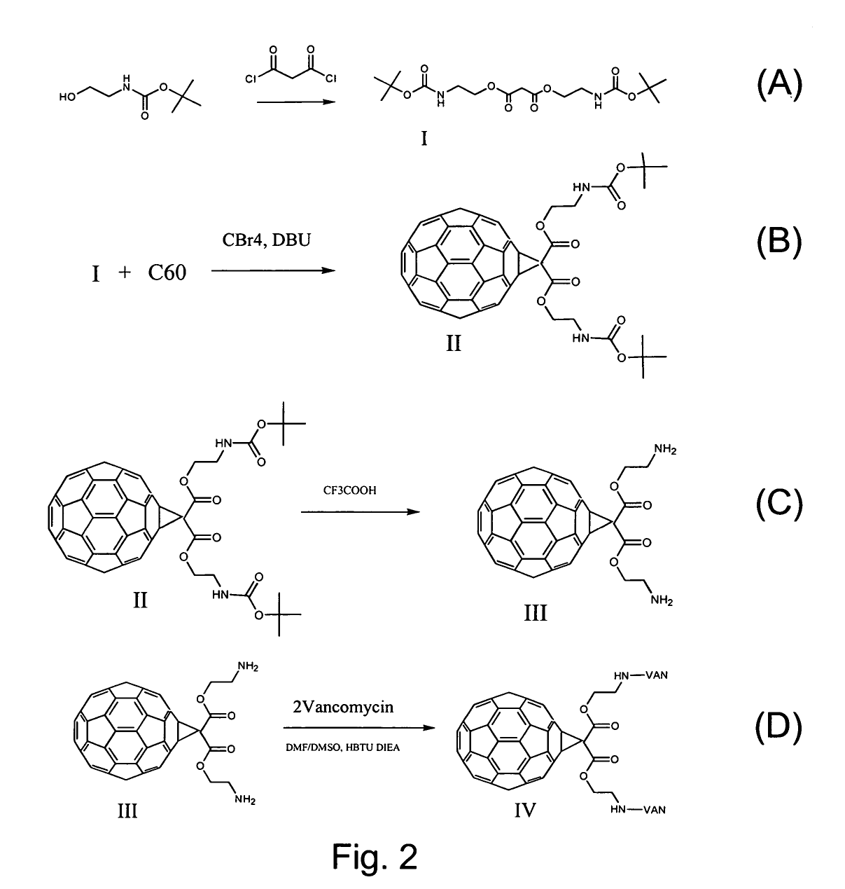

To condense vancomycin with C₆₀, an HBTU-mediated coupling protocol was used. This approach begins with the attachment of a linker containing an amino group to the fullerene. A malonate containing t-Boc-protected amino groups was synthesized. As shown in FIG. 2(A), two equivalents of tert-butyl N-(3-hydroxypropyl)carbamate, dissolved in anhydrous toluene, were then reacted with 1 equivalent of malonyl chloride in the presence of 2 equivalents of dry pyridine, to yield a malonate (I). The malonate (I) was then purified by flash chromatography on silica with a hexane/ethyl-acetate 1:1 mixture. Its structure was proven by ¹H and ¹³C NMR.

As shown in FIG. 2(B), the malonate (I) was then reacted with C₆₀ via the Bingel-type reaction in toluene, to produce (II), which was in turn hydrolyzed with trifluoroacetic acid, resulting in the desired amino derivative of C₆₀ (III). The structure of (III) was confirmed by ¹H NMR and MALDI-TOF MS.

For the last step, 1 equivalent of (III) was reacted with vancomycin (2.2 equivalents) in a DMF/DMSO solvent mixture. DIEA was used as a base and HBTU as a coupling agent. After reacting for 8 h. the fullerene-vancomycin conjugate (IV) was precipitated from the reaction mixture with acetonitrile. The product was washed several times with acetonitrile. The structure of (IV) was confirmed by MALDI TOF MS and ¹H NMR.

While preferred embodiments of this invention have been shown and described, modifications thereof can be made by one skilled in the art without departing from the spirit or teaching of this invention. The embodiments described herein are exemplary only and are not limiting. Many variations and modifications of the system and apparatus are possible and are within the scope of the invention. Accordingly, the scope of protection is not limited to the embodiments described herein, but is only limited by the claims which follow, the scope of which shall include all equivalents of the subject matter of the claims.

What is claimed is:

1. A fullerene-antibiotic conjugate including at least one antibiotic molecule per fullerene moiety.

2. The fullerene-antibiotic conjugate according to claim 1 wherein the fullerene comprises C₆₀.

3. The fullerene-antibiotic conjugate according to claim 2 wherein the antibiotic comprises vancomycin.

4. The fullerene-antibiotic conjugate according to claim 2 wherein the conjugate includes at least two antibiotic molecules per C₆₀ center.

5. The fullerene-antibiotic conjugate according to claim 2 wherein the conjugate includes at least three antibiotic molecules per C₆₀ center.

6. The conjugate according to claim 1 wherein the antibiotic is selected from the group consisting of penicillins, cephalosporins, quinolones, fluoroquinolones, macrolides, lincosamines, carbepenems, conobactams, aminoglycosides, glycopeptides, tetracyclines, sulfonamides, rifampin, oxazolidonones, and streptogramins.

7. The conjugate according to claim 1, further including a targeting agent comprising an antigen-binding site.

8. The conjugate according to claim 7 wherein the targeting agent is capable of binding to anthrax spores.

9. The conjugate according to claim 1, further including a targeting agent comprising a bone-targeting moiety.

10. An antibiotic treatment comprising an aerosol mist comprising the fullerene-antibiotic conjugate of claim 1.

11. A method for making a fullerene(C₆₀)-antibiotic conjugate, comprising:
a) synthesizing a linker precursor (I);
b) reacting the linker precursor (I) with C₆₀ via a Bingel-reaction, to produce a fullerene-linker conjugate (II);
c) hydrolyzing the fullerene-linker conjugate (II), resulting in a desired derivative of C₆₀ (III); and
d) reacting the derivative (II) with a desired antibiotic to produce a fullerene-antibiotic conjugate (IV).

12. The method according to claim 11 wherein the linker precursor is a malonate having t-Boc-protected amino groups.

13. The method according to claim 11 wherein the derivative made in step c) is an amino derivative.

14. The method according to claim 11 wherein the Bingel-reaction in step b) is carried out in toluene.

15. The method according to claim 11 wherein step c) is carried out using trifluoroacetic acid.

16. The method according to claim 11 wherein the step d) is carried out in a DMF/DMSO solvent mixture.

17. The method according to claim 11 wherein step d) is carried out using DIEA as a base and HBTU as a coupling agent.

18. The method according to claim 11 wherein the step e) is carried out using acetonitrile.

19. The method according to claim 11, further including precipitating a fullerene-antibiotic conjugate (IV) from the reaction mixture.

20. The method according to claim 19, further including the additional step of washing the precipitated a fullerene-antibiotic conjugate (IV).

21. The method according to claim 11, further including the step of incorporating the a fullerene-antibiotic conjugate (IV) into a pharmaceutical composition.

22. A method of killing a microorganism infecting a mammal, the method comprising contacting said microorganism with a fullerene-antibiotic conjugate including at least one antibiotic molecule per fullerene moiety.

23. A pharmaceutical composition comprising a fullerene-antibiotic conjugate including at least one antibiotic molecule per fullerene moiety, said conjugate being present in a pharmaceutically acceptable carrier.

24. A method of inhibiting the growth of a bacterial species in a human subject, comprising:
administering to a human subject having a bacterial infection or overgrowth a pharmaceutically acceptable composition containing fullerene-antibiotic conjugate in a dose effective to inhibit the growth of a bacterial species in the human subject.

25. The method of claim 24, wherein said fullerene-antibiotic conjugate comprises C₆₀ conjugated with an antibiotic is selected from the group consisting of penicillins, cephalosporins, quinolones, fluoroquinolones, macrolides, lincosamines, carbepenems, conobactams, aminoglycosides, glycopeptides, tetracyclines, sulfonamides, rifampin, oxazolidonones, and streptogramins.

26. The method of claim 18 wherein the administration is carried out by a technique selected from the group consisting of: non-systemic delivery routes, including colonic delivery routes, ingestive delivery routes, topical applications of cream, gel, or ointment, and systemic delivery routes, including inhalation, ingestion, injection, intravenous drip, implant, transdermal delivery routes, and transmucosal delivery routes.
